(19) United States
(12) Patent Application Publication
(10) Pub. No.: US 2022/0338686 A1
(43) Pub. Date: Oct. 27, 2022

(54) Sensing Devices

(71) Applicants: Brian S. Jolin, Chicago, IL (US); Cosmin D. Mclea, Chicago, IL (US); Renzo Francesco Giuliano Ruiz, Oranjestad (AW); Andrea Lombardo, Milano (IT)

(72) Inventors: Brian S. Jolin, Chicago, IL (US); Cosmin D. Mclea, Chicago, IL (US); Renzo Francesco Giuliano Ruiz, Oranjestad (AW); Andrea Lombardo, Milano (IT)

(21) Appl. No.: 17/693,064

(22) Filed: Mar. 11, 2022

Related U.S. Application Data
(63) Continuation of application No. PCT/US2020/050523, filed on Sep. 11, 2020.
(60) Provisional application No. 62/898,755, filed on Sep. 11, 2019.
(60) Provisional application No. 63/016,511, filed on Apr. 28, 2020.

(51) Int. Cl. A47K 10/48; G06F 3/03; H04W 4/38; G01S 17/08; G01S 7/4865; A61L 2/20

(52) U.S. Cl. CPC A47K 10/48; G06F 3/0304; H04W 4/38; G01S 17/08; G01S 7/4865; A61L 2/202; A61L 2202/14

(57) ABSTRACT

A hand dryer includes improved electronic features and internal and external sensors. The improved hand dryer provides useful sensors for public restrooms. In that regard, air quality sensors, smoke detectors and ozone sensors provided by the hand dryer are particularly useful. A portable sensor device has electronic features and internal and external sensors. The portable sensor can be used by a traveler to make a traveler aware of the contents of the air they will be breathing in a hotel room. The portable sensor has sensors providing other valuable information to the traveler and can monitor the security of the room when the traveler is absent.

This application is a continuation of PCT/US2020/050523 filed Sep. 11, 2020 which claims the benefit of U.S. Ser. No. 62/898,755 filed Sep. 11, 2019, and U.S. Ser. No. 63/016,511 filed Apr. 28, 2020.

### BACKGROUND

One embodiment of the invention described herein is an improved hand dryer. Hand dryers with electronic components are known as described in US Published Patent application 2008/0004963.

A hand dryer with an interactive display screen and sensors is disclosed in Published US Patent Application 2016/0256021. While the hand dryer in this application provides many sensors for data collection and display, the present inventors have recognized the desirability for a hand dryer to have more sensors and improved sensors.

Another embodiment of the invention described herein relates to a portable sensor device that provides environmental information to a user.

Business travelers are usually away from their rooms while on a business trip, leaving their expensive and confidential belongings when out at meetings or conferences. Vacationers are usually away from their rooms during vacation and leave valuable items unattended. The recent growth of private property rentals for millennial travelers creates a market of demand for better security and privacy while staying at a stranger's apartment or home. Air quality is a growing concern in the travel industry. While major hotels do take measures to ensure the quality of the air is optimal for their gas, small hotels and private properties may not have proper filtration systems in place for protecting air quality.

### SUMMARY

An exemplary first embodiment of the present invention provides a hand dryer having improved electronic features and internal and external sensors.

The improved hand dryer provides useful sensors for public restrooms particularly that serve many people such as in airports, stadiums, institutions, etc. Additionally, the improved hand dryer provides useful sensors for public restrooms that are located in hospitals, medical centers and laboratories. In that regard, air quality sensors, smoke detectors and ozone sensors provided by the hand dryer are particularly useful.

Some of the sensors provided on, in or in the vicinity of the hand dryer are:

### NFC/RFID Sensor

This sensor reads RFID of NFC readable card and mobile phones for check-in and check-out of employees. Also, this sensor could function as an authentication method for maintenance members to be granted access for on-site maintenance. The sensor can be a TEXAS INSTRUMENTS TRF7970A Transceiver IC such as described at https://www.mouser.com/new/texas-instruments/ti-trf7970a-transceiver-ic/. NFC is the technology used to contact the occasional user to download information, coupons.

### Time-of-Flight Sensor

The hand dryer can have two time-of-flight sensors. A hands time-of-flight sensor can be located on the bottom of the hand dryer to detect the presence of hands and to activate the blower, and a user time-of-flight sensor can be located on the front of the hand dryer to detect the presence of a person and to give accurate data on how long the person is standing there. The time-of-flight sensor can be an ST VL6180X proximity sensor, gesture and ambient light sensing (ALS) module as described at https://www.st.com/content/st_com/en/products/imaging-and-photonics-solutions/proximity-sensors/v16180x.html.

### Z-Wave Communication Module

The hand dryer can have a Z-wave communication module that uses a wireless communications protocol to connect smart devices and external sensors. For example, a Z-wave sensor can be installed in the toilet paper dispenser, alerting the facility manager when toilet paper is running out. The Z-wave sensor can be a ZGM130S SIP Module from Silicon Labs, described at https://www.silabs.com/products/wireless/mesh-networking/z-wave/modules/zgm130s-sip-module.

### Ozone Detector/Generator

The hand dryer can have an ozone generator to purify the air and remove bad odors and kill bacteria on the hand. The hand dryer can also have an ozone detector to precisely calculate the ozone generated and thereby avoid overexposure to people. The ozone sensor can be a 3SP-03-20 sensor from SPEC SENSORS, described at https://www.spec-sensors.com/product/ozone-sensor/. Alternately, the ozone sensor can be a model MHM501-00 ozone sensor from MURATA as described at https://eu.mouser.com/productdetail/murata-electronics/mhm501-00?qs=%2fha2pyfaduhgwqoxjatvgr5hokxk%252bkeldqmessebisa8wrummjtnew%3d%3d. Ozone could also be used as the most effective way of sterilizing the inner part of the hand dryer, avoiding bacteria proliferation. Ozone levels must be monitored to avoid reaching a level that could be uncomfortable for people.

### LTE/Wi-Fi Module

The hand dryer can have an LTE/Wi-Fi module giving the hand-dryer ability to connect to either a Wi-Fi or a cellular network, described at https://y1cj3stn5fbwhv73k0ipk1eg-wpengine.netdna-ssl.com/wp-content/uploads/2019/02/telit_me910g1_datasheet.pdf. The hand dryer can primarily rely on Wi-Fi to receive data and send out warnings. This type of connection is free, has an abundant bandwidth and it is extremely reliable in public places, such as, airports, offices, stations, etc. Where Wi-Fi is absent or is more difficult to gain access, an LTE connection can be included. A TELIT module HE910 CAT1 device can be used or similar. Alternatively, cable is another way to get the hand dryer on line.

### Air Quality Sensor

The hand dryer can have an air quality sensor used to detect dangerous toxins and particles in the air. PM10/PM 2.5 are the classical polluting indicators. They could be a very precise and sensitive smoke detectors.

### Blower Controller

The hand dryer can have an onboard blower controller that controls a blower motor which drives the blower fan. The fan speed and ease on/off can be controlled through the onboard blower controller. This will give the ability to turn on and off the blower motor and adjust the sound of the hand-dryer as well.

### BLUETOOTH

For connection to the regular user, if a BLUETOOTH application installed, BLUETOOTH is the primary way of the user communicating with the hand dryer.

### Gesture Sensor

TEXAS INSTRUMENTS mmWave technology can be used for gesture. MmWave is a sensing technology for the detection of objects and for providing the range, velocity and angle of these objects. It is a contactless-technology which operates in the spectrum between 30 Ghz-300 Ghz. Due to the technology's use of small wavelengths it can provide sub-mm range accuracy and is able to penetrate certain materials such as plastic, drywall, clothing, and is impervious to environmental conditions, such as rain, fog, dust and snow.

Accordingly, the sensor could be inside the hand dryer with no exposed part, making it nearly impossible to tamper with, and will not be Influenced by moisture or water droplets.

### Presence Sensor

A presence sensor detects the presence of a user in front of the hand dryer. To obtain precise information, not influenced by light and clothes, TOF (time-of-flight) sensors, such as the VL6180X, can be used. The VL6180X is the latest product based on ST's FLIGHTSENSE™ technology. This technology allows absolute distance to be measured independent of target reflectance. Instead of estimating the distance by measuring the amount of light reflected back from the object (which is significantly influenced by color and surface), the VL6180X precisely measures the time the light takes to travel to the nearest object and reflect back to the sensor (time-of-flight).

### User Classification

Sensor systems can be used to discriminate the type of users in front of the device, and are able to distinguish with good reliability gender and age.

Other detailed information can be collected by these sensors, are, for example:
expression estimation
facial pose estimation
gaze estimation
blink estimation
hand detection
human body detection

One system which can be used is the OMRON HVC-P device, based on OKAO™ vision image sensing technology.

### Electronic Nose—Urine Detection, Bad Smell

Normal air quality sensors are sensitive to very specific chemicals. The sense of a bad smell it is not detectable directly from a single sensor. Papers are available for electronic noses. Array of sensors, each one capable of a single detection. For a trained neural network, processing the data collected by each element of the array, the network can determine a very specific situation. Efficiency and reliability could be different in different countries, due to different eating habits.

### Smoke Detectors

Classical smoke detectors could be inserted in the hand dryer.

### Microphone

A microphone can be provided in the hand dryer for a basic two way communicating system. The microphone could be activated, to be activated when a panic button is pressed. Information could be more valuable if a beam forming process is created using a microphone array, instead of a single device.

### Ecosystem—Radio Link 1/3—Radio Link 2/3—Radio Link 2/3

The hand dryer can be the technology hub inside the restroom. Like every hub, it should be linked with other devices. Building an ecosystem, the link technology can be chosen considering the most used link technology adopted in commercial devices that could be added to the eco system. Considering the variety of devices, the link technology could take into consideration a covering range of 100 meters or more and the possibility to run on batteries.

Custom Radio Protocol UB GIGA

Datagram are smaller, extremely low power, long range, and unidirectional.

Some of the actuators and devices provided in the hand dryer are:

### Motor Drive

The main function of the hand dryer is to generate the air flow. Driving the motor electronically, makes it possible to control speed and noise.

### Ozone Generator

Sanitizing with UV with an ozone generator can produce the required effect, with less power and more effectiveness. The generator can be equipped with the ozone sensor to verify that the level of ozone is within preset desired margins.

### CPU or Single Board Computer

Video playback can be provided as desired through a hand dryer screen. For playback of a 1024×768 full color video, a SOM with the capacity of playing back compressed video, could be DIGI CONNECTCORE 6+,
scalable CORTEX-A9 multi-core performance
independent CORTEX-M0+/CORTEX-M4 MICROCONTROLLER ASSIST™ subsystem
cost-effective, reliable, low-profile surface-mount module form factor
pre-certified 802.11a/b/g/n/ac and BLUETOOTH 4.2
smart power management architecture with PMIC
ANDROID and YOCTO project LINUX software platform support
reliable design with IEC 60068 and halt verification
dedicated on-module security+authentication controller

An exemplary second embodiment of the present invention provides a portable sensor device having electronic features and internal and external sensors.

The exemplary embodiment of the invention can be used by a traveler to make a traveler aware of the contents of the air they will be breathing in a hotel room.

The exemplary embodiment of the invention can also provide sensors providing other valuable information to the traveler and can monitor the security of the room when the traveler is absent.

The portable sensor device provides useful sensors for use at home or by travelers that provides useful sensors and a camera for monitoring a user's environment, such as a hotel room for safety and security. In that regard, the device includes a housing that encloses one or more air quality sensors, a pressure sensor, a radar motion sensor, a climate sensor, a radio frequency sensor, a motion sensor, first alert sensor, GPS locator. The housing can also contain Bluetooth for pairing, an HD camera, a battery, and built in memory and control electronics.

Some of the sensors provided on, in or in the vicinity of the device are:

### Radio Frequency Sensor

This sensor detects unauthorized wireless cameras or microphones or other spy or “bug” devices that could be present surreptitiously in a room. One example is the Anself CC308+ multi-functional Full-RF Wireless Signal Radio Detector Camera Auto-detection Tracer Finder 1 mHz-6 in a hand-held model. The functional components of such a device could be incorporated into the unit. See, for example https://balmahome.com/product/the-best-bug-detector-hidden-camera-and-microphones-detector/?qclid=EAIaIQobChMIhIWY0LX86AIVUvDACh3G9A1HEAkYBiABEgKFwPD_BwE.

mmWave Sensor

This sensor uses millimeter wave radar technology to create a mapping of the room and detect movements throughout the area. The sensor detects where patrons are within the room and when they enter or leave. The mmWave sensor can be from Texas Instruments, described at http://www.ti.com/sensors/mmwave/overview.html. It can be a TI AWR1642 mmWave sensor.

### Time-of-Flight Sensor

This sensor can be used to detect the presence of a person and to give accurate data on how long the person is in the room. The time-of-flight sensor can be an ST VL6180X proximity sensor as described at https://www.st.com/content/st_com/en/products/imaging-and-photonics-solutions/proximity-sensors/vI6180x.html.

The VL6180X is the latest product based on ST's FLIGHTSENSE™ technology. This technology allows absolute distance to be measured independent of target reflectance. Instead of estimating the distance by measuring the amount of light reflected back from the object (which is significantly influenced by color and surface), the VL6180X precisely measures the time the light takes to travel to the nearest object and reflect back to the sensor (time-of-flight).

### LTE/Wi-Fi Module

The device can have an LTE/Wi-Fi module giving the device the ability to connect to either a Wi-Fi or a cellular network, described at https://y1cj3stn5fbwhv73k0ipk1eg-wpengine.netdna-ssl.com/wp-content/uploads/2019/02/telit_me910g1_datasheet.pdf. The device can primarily rely on Wi-Fi to receive data, record data and/or send out warnings. This type of connection is free, has an abundant bandwidth and it is extremely reliable in public places, such as, airports, offices, stations, etc. Where Wi-Fi is absent or is more difficult to gain access, an LTE connection can be included. A TELIT module HE910 CAT1 device can be used or similar. Alternatively, cable is another way to get the device on line.

### Air Quality Sensor

The device can have an air quality sensor used to detect dangerous toxins and particles in the air. PM10/PM 2.5 are the classical polluting indicators. They could be a very precise and sensitive smoke detectors.

### Climate Sensor

The device can also include temperature, humidity and air pressure sensors.

### BLUETOOTH

For connection to the regular user, if a BLUETOOTH application installed, BLUETOOTH is the primary way of the user communicating with the device.

### Smoke Detector

Classical smoke detectors could be inserted in the device.

### Microphone

A microphone can be provided in the device for a basic two way communicating between a user and the device using voice recognition software. The microphone can also be utilized for voice communication through the device to another person. The microphone can be used as a sound detector to initiate camera recording of the room.

### Loudspeaker

The device can include a loudspeaker for audible communication from the device to a user, for entertainment such as music, and alarms, such as wake-up alarm or an emergency alarm, such as for the presence of smoke, toxins, or an intruder.

### CPU or Single Board Computer

scalable CORTEX-A9 multi-core performance
independent CORTEX-M0+/CORTEX-M4 MICROCONTROLLER ASSIST™ subsystem
cost-effective, reliable, low-profile surface-mount module form factor pre-certified 802.11a/b/g/n/ac and BLUETOOTH 4.2
smart power management architecture with PMIC
ANDROID and YOCTO project LINUX software platform support
reliable design with IEC 60068 and halt verification
dedicated on-module security+authentication controller

### Camera

A 1080p HD camera can be provided, communicating through an imaging processing board to monitor the room when the traveler is not present. The still or video images can be recorded in the device memory or uploaded to an external database in real time to capture any unauthorized activity in the traveler's room, such as theft, when the traveler is not present. Either the time-of-flight sensor or the mmWave sensor can trigger the camera to start recording if a person is present in the room.

### Camera Lens Detector

A strobing RGB LED light can be used with the CPU and a filtered image of the strobing RGB LED light to detect small reflections in the recording by the HD camera to identify a lens of a surreptitiously placed camera in a hotel room or the like. The HD camera can scan the room while the RGB LED light is strobing and the CPU can identify the reflection of a camera lens of a hidden camera. An example of a device which uses this technology is the Spy Finder Pro Hidden Camera Detector, available from Brickhouse Security at https://www.brickhousesecurity.com/counter-surveillance/spy-finder/ Manhattan, NY, N.Y.

The exemplary embodiment of the device according to the invention provides safety and security of a traveler who is in a room, or remote from the room, including the person of the traveler or the traveler's belongings.

mmWave Sensors

This sensor uses millimeter wave radar technology to create a mapping of the room and detect movements throughout the area. The sensor detects where patrons are within the room and when they enter or leave. The mmWave sensor can be from Texas Instruments, described at http://www.ti.com/sensors/mmwave/overview.html.

Numerous other advantages and features of the present invention will be become readily apparent from the following detailed description of the invention and the embodiments thereof, and from the accompanying drawings.

### BRIEF DESCRIPTION OF THE DRAWINGS

FIG. 1 is a schematic elevation view of a hand dryer according to the invention;

FIG. 2 is a block diagram of the system of the invention; and

FIG. 3 is a user display screen responding to the system of FIG. 2.

FIG. 4 is a block diagram of an exemplary portable sensor device of the invention;

FIG. 5 is a schematic diagram of a control board for the exemplary portable sensor device of FIG. 4;

FIG. 6 is a graphic dashboard of a remote device responding to camera data from the portable sensor device;

FIG. 7 is a graphic dashboard of a remote device responding to air quality data from the portable sensor device;

FIG. 8 is a graphic dashboard view of a remote device responding to a camera lens of a hidden camera; and

FIG. 9 perspective view of the exemplary portable sensor device of FIG. 4.

### DETAILED DESCRIPTION

While this invention is susceptible of embodiment in many different forms, there are shown in the drawings, and will be described herein in detail, specific embodiments thereof with the understanding that the present disclosure is to be considered as an exemplification of the principles of the invention and is not intended to limit the invention to the specific embodiments illustrated.

This application incorporates by reference U.S. Pat. No. 10,342,398; US Published Application 2008/0004963; PCT/US2020/050523 filed Sep. 11, 2020; U.S. Ser. No. 62/898,755 filed Sep. 11, 2019; and U.S. Ser. No. 63/016,511 filed Apr. 28, 2020.

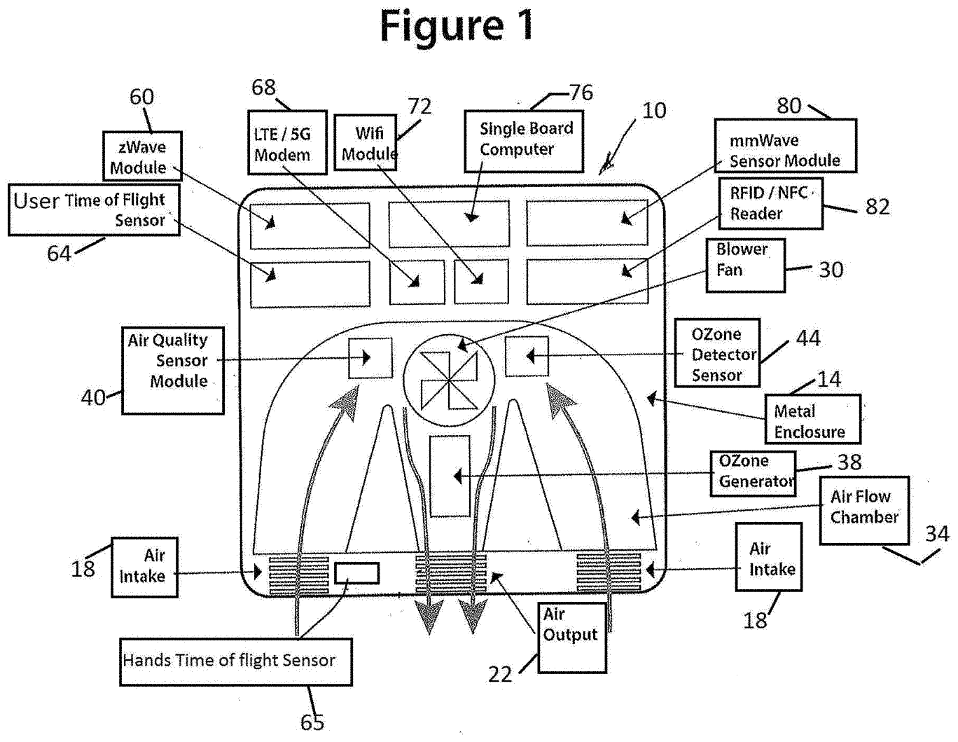

FIG. 1 illustrates in schematic form, a hand dryer 10 having a housing 14, such as a metal enclosure, with an air intake 18 and an air outlet 22. A blower fan 30 is arranged in an air chamber 34 between the air intake 18 and the air outlet 22 and is powered to pressurize air from the air intake 18 and deliver air at a velocity out of the air outlet 22 in order to dry hands held beneath the air outlet. An air heater (not shown) can also be provided within the air chamber in order to provide heated air to dry hands.

An ozone generator 38 is located within the air chamber 34, and is used to purify the air and remove bad odors and kill bacteria on the hands. The ozone generator can be arranged in the outlet to treat the air that dries hands. An air quality sensor 40 and an ozone detector sensor 44 can be arranged in the air flow chamber 34 to check air for contaminants and ozone respectively. The ozone sensor precisely measures the concentration of ozone and the computer controls the ozone generator such as to avoid a concentration that may be detrimental to occupants of the restroom. The ozone sensor can be an 3SP-03-20 sensor from SPEC SENSORS, described at https://www.spec-sensors.com/product/ozone-sensor/. Alternately, the ozone sensor can be a model MHM501-00 ozone sensor from MURATA as described at https://eu.mouser.com/productdetail/murata-electronics/mhm501-00?qs=%2fha2pyfaduhgwqoxjatvgr5hokx%252bkeldqmessebisa8wrummjtnew%3d%3d.

Within the housing 14 above the air flow chamber, a number of electronic sensors and devices are arranged.

A Z-wave module 60 uses a wireless communications protocol to connect smart devices and external sensors. For example a sensor can be installed in the toilet paper dispenser, alerting the facility manager when toilet paper is running out by the Z-wave wireless protocol. The Z-wave sensor can be a ZGM130S SIP Module from SILICON LABS, described at https://www.silabs.com/products/wireless/mesh-networking/z-wave/modules/zgm130s-sip-module.

The hand dryer can have two time of flight sensors 64. One time-of-flight sensor can be located on the bottom of the hand dryer to detect hands and activate the blower, and another time-of-flight sensor can be located on the front of the hand dryer to detect the presence of the person to give accurate data on how long the person is standing there. The time-of-flight sensor can be an ST VL6180X proximity sensor, gesture and ambient light sensing (ALS) module as described at https://www.st.com/content/st_com/en/products/imaging-and-photonics-solutions/proximity-sensors/v16180x.html.

An LTE/5G modem 68 gives the hand-dryer the ability to connect to either a Wi-Fi or a cellular network, described at https://y1cj3stn5fbwhv73k0ipk1eg-wpengine.netdna-ssl.com/wp-content/uploads/2019/02/telit_me910g1_datasheet.pdf.

A WiFi module 72 is provided in the housing for users.

A single board computer 76 is mounted within the housing and receives data from the sensors, controls devices and collects, stores and transmits sensor data.

An mmWave sensor module 80 uses millimeter wave radar technology to create a mapping of the room and to detect movements throughout the area, detecting where patrons are within the room and when they enter or leave. This sensor can map out the restroom with it creating the ability to count people passing through its field of view in the restroom. When the restroom is mapped out with their stalls etc., which stall the persons have used can be detected and their behavior can be detected when entering and exiting the facility. The mmWave sensor can be from TEXAS INSTRUMENTS, described at http://www.ti.com/sensors/mmwave/overview.html.

An RFID/NFC reader 82 reads RFID of NFC readable card and mobile phones for check-in and check-out of employees. Also, this sensor could function as an authentication method for maintenance members to be granted access for on-site maintenance. The sensor can be a TEXAS INSTRUMENTS TRF7970A Transceiver IC such as described at https://www.mouser.com/new/texas-instruments/ti-trf7970a-transceiver-ic/.

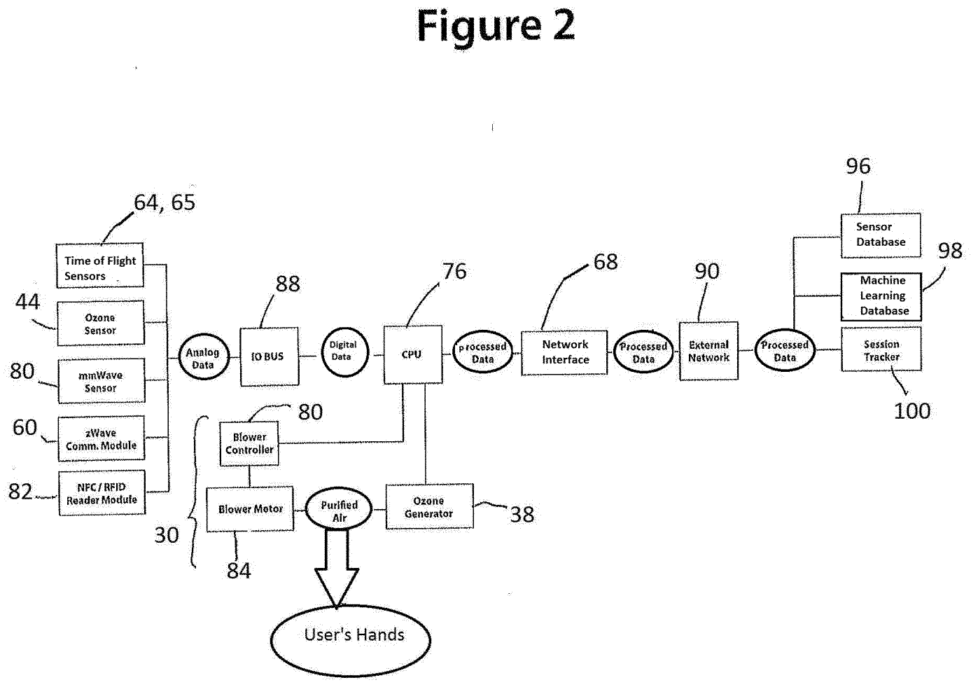

FIG. 2 illustrates the time-of-flight sensors 64, 65 the ozone sensor 44, the mmWave sensor 80, the Z-Wave sensor 60 and the NFC/RFID reader module 82 provide analog data to the 10 Bus 88 which outputs digital data to the CPU or single board computer 76. Based on some of these sensor signals, the computer 76 controls a blower controller 80 which controls a blower motor 84. The computer 76 also controls the operation of the ozone generator 38. Otherwise the computer collects, stores and transmits data to an external network 90.

The computer 76 supplies processed data to a network interface such as the LTE/5G modem 68 to an external network 90 which provides data to a sensor database 96, a machine learning database 98 and a session tracker 100.

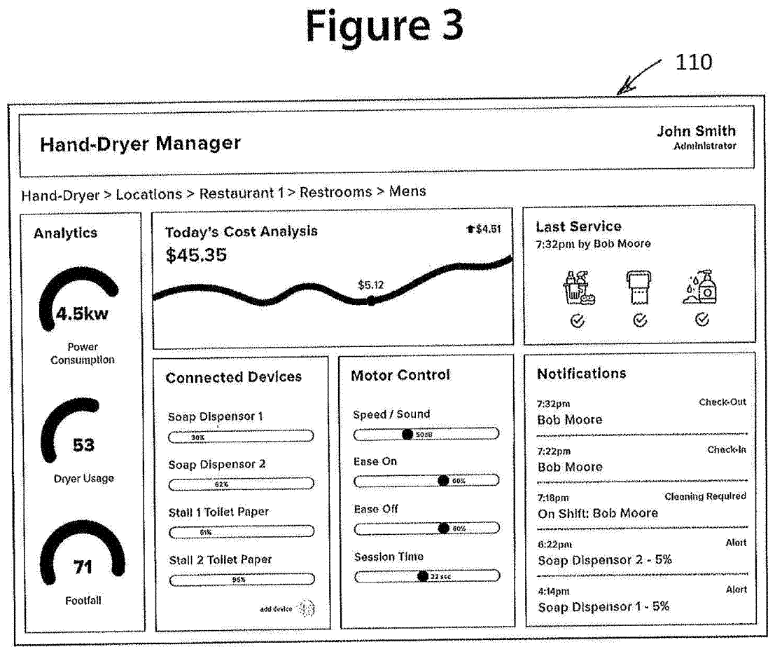

The session tracker 100 can be in the form of a user interface 110 as shown in FIG. 3. The sensors provide data which can be displayed on the interface, in the form of a viewing screen, such as a daily power consumption, number of dryer uses, number of people passing by the dryer, a cost analysis, maintenance or fill status of soap dispensers, toilet paper rolls, control functions of the blower motor, alerts, service recommendations and employee check-ins and check-outs.

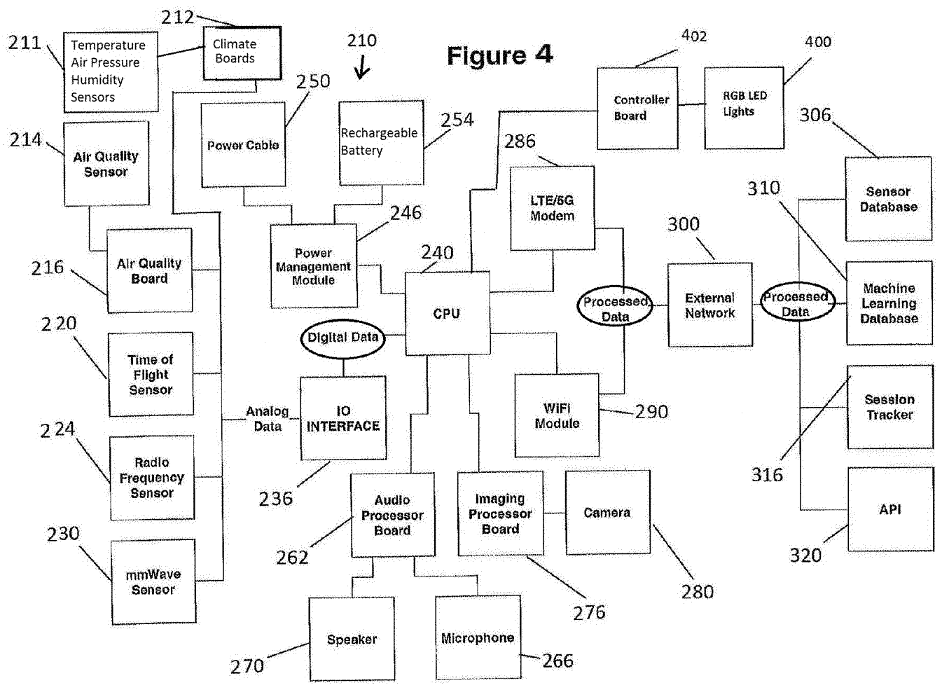

FIG. 4 illustrates in schematic form, a second embodiment of the invention, a portable sensor device 210. The device 210 includes a variety of sensors, including climate sensors 211 in signal communication with a climate board 212, an air quality sensor 214 in signal communication with an air quality module or board 216, a time-of-flight sensor 220, a radio frequency sensor 224 and an mmWave sensor 230. The time of flight sensor 220 can project a “3D” image and can detect movement as well as people passing in front of the unit. The radio frequency sensor 224 detects if there are any devices emitting radio frequencies from wireless cameras or recording devices (“bugs”) inside the room. The mmWave sensor (or radio motion sensor) creates a radar field of the room and can track movement around the room. The air quality sensors 214 include sensors that can measure pollutants in the air and also temperature, humidity and pressure. All these sensors output analog data to an IO interface 36 which communicates digital data to a CPU 240. A power management module 46 is signal connected to the CPU 240. The power management module 246 receives power from either a power cable 250 or a rechargeable battery 254.

An audio processor board 262 is signal connected to the CPU 240. The audio processing board receives input from a microphone 266 and outputs audio signal to a loudspeaker 270. The microphone can be used to communicate commands to the device CPU 240 via voice recognition software. The loudspeaker can be used to audibly communicate information to the user.

An imaging processing board 276 is signal connected to the CPU 240 and receives a video signal from a 1080p HD camera 280. The 1080p HD camera can monitor the room when the user is not present. Still or video images can be recorded in the device memory and/or uploaded to an external database in real time to capture any unauthorized activity in the user's room, such as an unauthorized intrusion or theft, when the user is not present. Either the time-of-flight sensor 220 or the mmWave sensor 230 can trigger the camera to start recording if a person is present in the room.

The microphone 266 can also be used as a sound detector to detect sound in the room to actuate the camera 280 via the audio processing board and the CPU to record activity in the room.

The CPU 240 receives and transmits signals between an LTE/5G modem 286 and a Wi-Fi module 290. The modem 286 and the Wi-Fi 290 communicate process data to and from an external network 300. The external network 300 exports and imports processed data such as to/from a sensor database 306, a machine learning database 310, a session tracker 316, and an API 320.

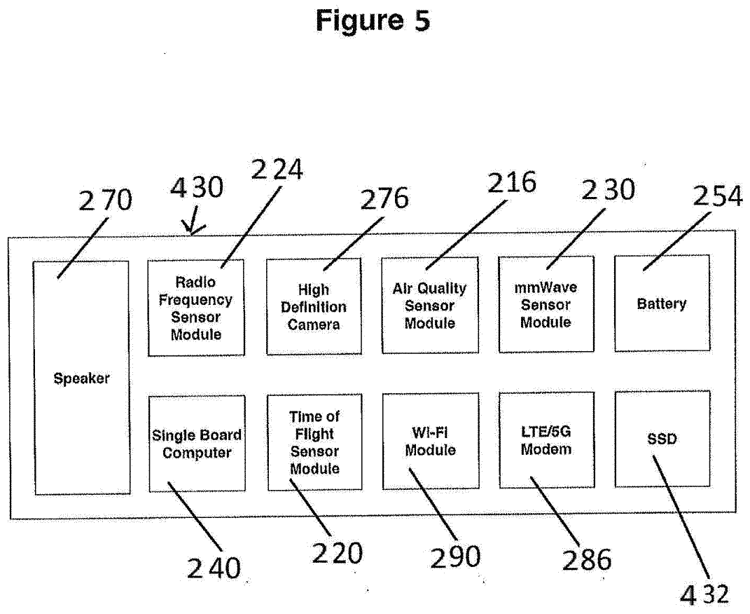

A strobing RGB LED light 400 is connected to a control board 402. The CPU 240 uses a filtered image of the strobing RGB LED light to detect small reflections in the recording by the HD camera 280 to identify a lens of a surreptitiously placed camera in a hotel room or the like. The HD camera can scan the room while the RGB LED light is strobing and the CPU 240 can identify the reflection of a camera lens of a hidden camera. The results of the scan can be displayed on a device, such as a desk top or laptop computer, or tablet, as shown in FIG. 5, or on a smart phone. An example of a device which uses this technology is the Spy Finder Pro Hidden Camera Detector, available from Brickhouse Security at https://www.brickhousesecurity.com/counter-surveillance/spy-finder/ Manhattan, NY, N.Y.

FIG. 5 illustrates a control board 430 for the device 210. The control board is an effective way to connect up the various sensors and modules of the device 210. If the various sensors are not part of their respective boards or modules, the sensors can be mounted separately from the board 430. The SSD 432 is a slot for micro-SSD cards.

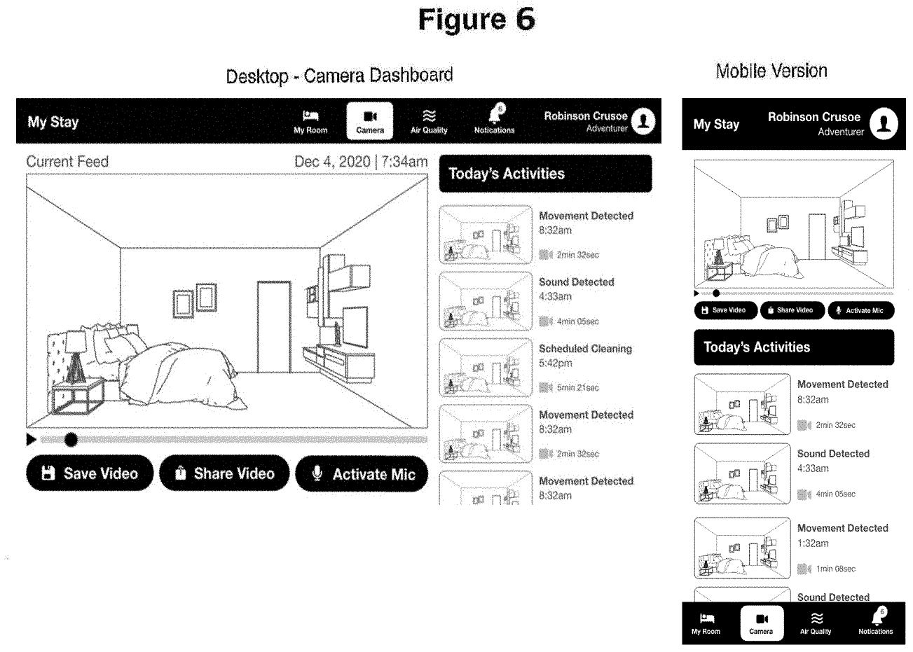

FIG. 6 is a camera view dashboard of images or video captured by the device. This dashboard can be displayed remotely from the device on a smartphone, on a desktop computer monitor or on a laptop. On the left in this figure the desktop or laptop display is shown. and on the right the smartphone display is shown. The dashboard can keep a running record of events that occur in the room a video clips of those events. The user can select any video clip or replay.

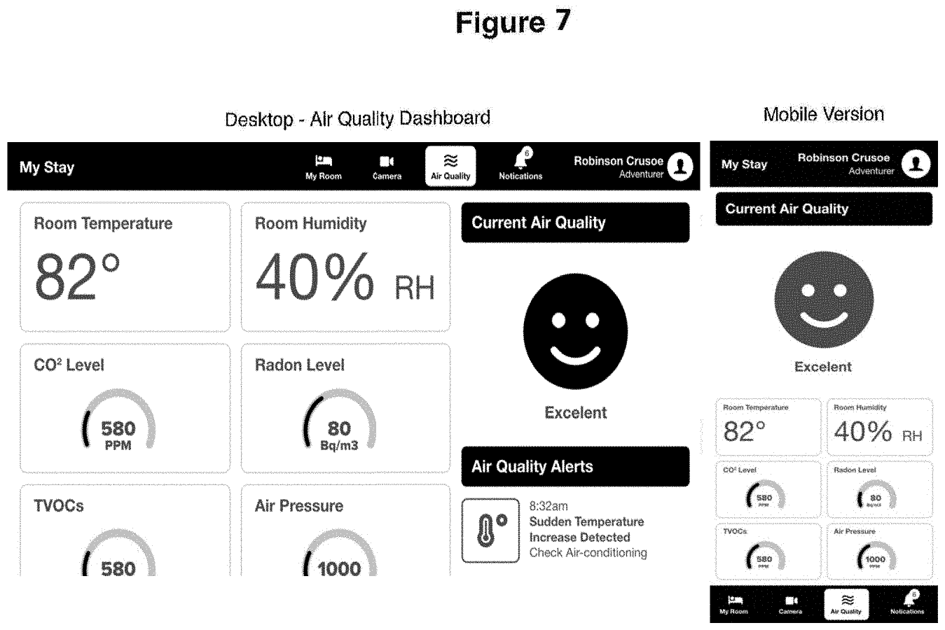

FIG. 7 is an air quality dashboard captured by the device air quality sensors and/or other sensors. This dashboard can be displayed remotely from the device on a smartphone, on a desktop computer monitor or on a laptop. On the left in this figure the desktop or laptop display is shown, and on the right the smartphone display is shown. The dashboard can keep a running record of events that occur in the room.

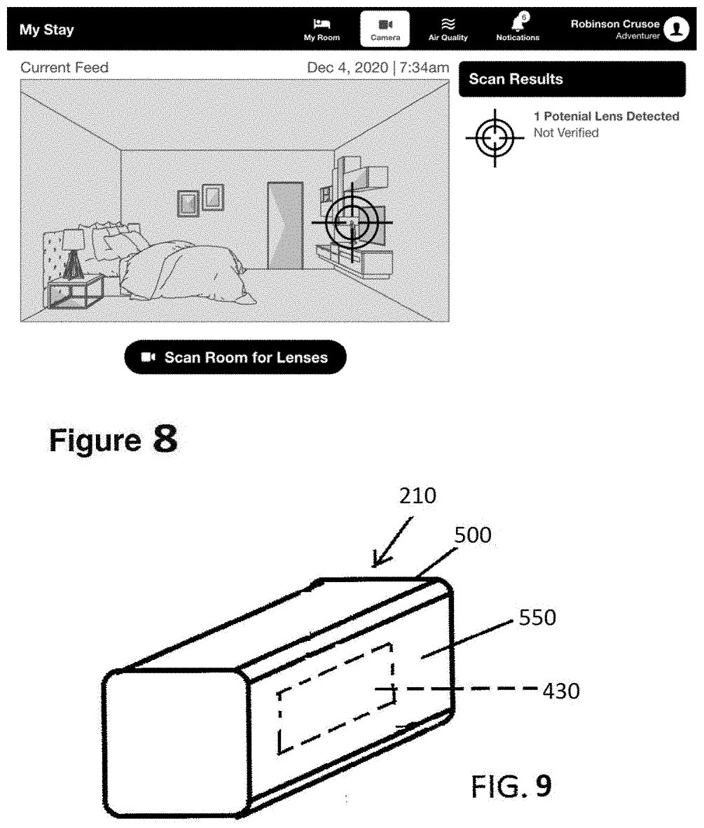

FIG. 8 is hidden camera dashboard captured by the device camera lens sensor using the RGB LED light 400, control board 402, CPU 240 and the HD camera 280. This dashboard can be displayed remotely from the device on a smartphone, on a desktop computer monitor or on a laptop. A desktop or laptop display is shown. The display indicates that the sensor has detected a surreptitious camera lens from a hidden camera.

FIG. 9 is a perspective view of the device. The device includes a box-like housing 500 that encloses the sensors and electronics of the device. The box-like housing can measure about 7 inches width, 2.5 inches depth and 2.5 inches height. The housing can have a user interface and/or the electronics which can communicate with a user using vice recognition, a handheld remote control, a smart phone, a web browser on a desktop or laptop computer. The board 430 is shown mounted on a front side off the housing. The sensors can also be mounted on a front side of the housing. The board 430 and sensors can be covered by a permeable front cover 550.

The housing can be a metal or plastic enclosure or made from other suitable material.

The climate sensors 211 can be arranged to monitor ambient conditions surrounding the device 210.

The air quality sensor 214 can be arranged to monitor air in the environment of the device.

The radio frequency sensor can be configured to sense any surreptitiously planted spying devices.

The time of flight sensor 220 is arranged to detect the presence of a person in a room. The time-of-flight sensor can be an ST VL6180X as described at https://www.st.com/content/st_com/en/products/imaging-and-photonics-solutions/proximity-sensors/v16180x.html.

The LTE/5G modem 286 gives the device the ability to connect to either a Wi-Fi or a cellular network, described at https://y1cj3stn5fbwhv73k0ipk1eg-wpengine.netdna-ssl.com/wp-content/uploads/2019/02/telit_me910g1_datasheet.pdf.

The WiFi module 290 is provided in the housing for users to upload data or download data. WiFi is not available everywhere so embedded cellular data allows a user to insert a local SIM card to have constant connectivity or sync later when the device is offline.

The single board computer 240 is mounted within the housing and receives data from the sensors, controls devices and collects, stores and transmits sensor data.

The mmWave sensor module 320 uses millimeter wave radar technology to create a mapping of the room and to detect movements throughout the room, detecting where people are within the room and when they enter or leave. The mmWave sensor can be from TEXAS INSTRUMENTS, described at http://www.ti.com/sensors/mmwave/overview.html.

The session tracker 416 can be in the form of a user interface with user input and a viewing screen. The sensors provide data which can be displayed on the interface, in the form of the viewing screen, such as air quality and people who enter the room. The viewing screen can be on a desktop computer, a laptop computer or a smartphone or other mobile device.

The machine learning would be to process data and create algorithms to increase the efficiency of the unit's features. For example, in cities where there are higher rating of pollution that different units have detected, the system would increase the limit of tolerable air pollution before sending an alert.

From the foregoing, it will be observed that numerous variations and modifications may be effected without departing from the spirit and scope of the invention. It is to be understood that no limitation with respect to the specific apparatus illustrated herein is intended or should be inferred.

1. A hand dryer comprising:
a housing,
an air blower in the housing and arranged to deliver air through an outlet of the housing out of the housing to dry hands;
an ozone generator within the housing, providing ozone to the air delivered through the outlet;
an ozone sensor for sensing the concentration of ozone in either the incoming air through the housing intake or the delivered air out of the outlet; and
a computer connected to the ozone generator and the ozone sensor, the computer turning off, or diminishing the output of, the ozone generator when the concentration of ozone reaches a pre-selected limit.

2. The hand dryer according to claim 1, further comprising a hands time-of-flight sensor mounted to the housing for detecting the presence of hands beneath the outlet, the computer connected to the hands time-of-flight sensor and to the air blower, the computer tuning on the air blower when hands are present.

3. The hand dryer according to claim 1, further comprising a user time-of-flight sensor for determining the presence of and duration of a user in front of the hand dryer, the computer connected to user hands time-of-flight sensor and collecting data on the presence of users in front of the hand dryer.

4. The hand dryer according to claim 1, further comprising an air quality sensor mounted to the housing and signal-connected to the computer, data on air quality collected by the computer.

5. The hand dryer according to claim 1, further comprising a WI-Fi module within the housing, communicating between user's devices and an external network.

6. The hand dryer according to claim 1, further comprising a Z-wave module communication with remote sensors and the computer, the computer collecting data from the remote sensors.

7. The hand dryer according to claim 1, further comprising a LTE/5G modem within the housing, communicating between the computer and an external network.

8. The hand dryer according to claim 1, further comprising a BLUETOOTH capable of signal-connection between the computer and a user's held device.

9. The hand dryer according to claim 1, further comprising an mmWave sensor module using millimeter wave radar technology creating a mapping of the room and detecting movements throughout the room, detecting where users are within the room and when they enter or leave.

10. The hand dryer according to claim 1, further comprising an RFID/NFC reader that reads RFID of NFC readable card and mobile phones.

11. The hand dryer according to claim 1, further comprising:
a user time-of-flight sensor for determining the presence of and duration of a user in front of the dryer and communicating with the computer for collecting presence and duration data;
an air quality sensor mounted to the housing and signal-connected to the computer for collecting air quality data;
a WI-Fi module within the housing;
a Z-wave module communication with remote sensors and the computer for collecting data from the remote sensors;
an LTE/5G modem within the housing;
a BLUETOOTH capable of signal-connection between the computer and a user's held device;
an mmWave sensor module using millimeter wave radar technology creating a mapping of the room and detecting movements throughout the room, detecting where users are within the room and when they enter or leave; and
an RFID/NFC reader that reads RFID of NFC readable card and mobile phones.

12. (canceled)

13. (canceled)

14. (canceled)

15. The hand dryer according to claim 22, further comprising an air quality sensor mounted to the housing and signal-connected to the computer to collect data on air quality.

16. The hand dryer according to claim 22, further comprising a WI-Fi module within the housing.

17. The hand dryer according to claim 22, further comprising a Z-wave module communication with remote sensors and with the computer to collect data from the remote sensors.

18. The hand dryer according to claim 22, further comprising a LTE/5G modem within the housing to communicate between the computer and an external network.

19. The hand dryer according to claim 22, further comprising a BLUETOOTH capable connection between the hand dryer and a user's hand held device.

20. (canceled)

21. (canceled)

22. A hand dryer comprising:
a housing,
an air blower in the housing and arranged to deliver air through an outlet of the housing out of the housing to dry hands;
an mmWave sensor module using millimeter wave radar technology creating a mapping of the room and detecting movements throughout the room, detecting where users are within the room and when they enter or leave; and
a computer connected to the mmWave sensor for collecting and storing data on where users are within the room and when they enter or leave.

23. A hand dryer comprising:
a housing,
an air blower in the housing and arranged to deliver air through an outlet of the housing out of the housing to dry hands;
remote sensors; and
a Z-wave module communication with the remote sensors; and
a computer connected to the Z-wave module for collecting and storing data on the remote sensors.

24.-42. (canceled)

43. The hand dryer according to claim 23, further comprising a user time-of-flight sensor for determining the presence of and duration of a user in front of the hand dryer, the computer connected to user hands time-of-flight sensor and collecting data on the presence of users in front of the hand dryer.
